(19) United States
(12) Patent Application Publication
(10) Pub. No.: US 2023/0024652 A1
(43) Pub. Date: Jan. 26, 2023

(54) PHARMACEUTICAL COMPOSITION COMPRISING SMALL OCTOPUS-DERIVED PEPTIDE FOR TREATMENT OF INFLAMMATORY DISEASE

(71) Applicant: NATIONAL MARINE BIODIVERSITY INSTITUTE OF KOREA, Chungcheongnam-do (KR)

(72) Inventors: Ilson HWANG, Chungcheongnam-do (KR); Bo Mi RYU, Jeju-do (KR)

(21) Appl. No.: 17/781,669

(22) PCT Filed: Jan. 22, 2021
(86) PCT No.: PCT/KR2021/000925

§ 371 Date: Jun. 1, 2022

(30) Foreign Application Priority Data
Jan. 22, 2020 (KR) 10-2020-0008336

(51) Int. Cl. A61K 38/17; A61K 38/10; A61P 29/00

(52) U.S. Cl. CPC A61K 38/1767; A61K 38/10; A61P 29/00

(57) ABSTRACT

The present invention relates to a pharmaceutical composition for treating inflammatory disease containing a peptide derived from Octopus minor. The novel antibacterial peptide or fragment thereof according to the present invention may efficiently inhibit pro-inflammatory cytokines and chemokines responsible for inflammatory responses in LPS-activated macrophages, and thus may be effectively used to prevent or treat inflammatory disease.

### TECHNICAL FIELD

The present invention relates to a pharmaceutical composition for treating inflammatory disease containing a peptide derived from Octopus minor, and more particularly, to a pharmaceutical composition for preventing or treating inflammatory disease containing, as an active ingredient, the Octopus minor-derived peptide Octominin that exhibits an anti-inflammatory effect on macrophages.

### BACKGROUND ART

Marine ecosystems represent a rich source of structurally unique and novel bioactive compounds from the perspective of potential therapeutic agents. Among natural marine products, peptides are important bioactive natural products which are abundantly found in many marine organisms. Recently, considerable attention has been focused on identifying bioactive marine peptides because of their novel chemical and diverse biological properties. Specifically, peptides derived from marine organisms having physiologically active properties such as ROS scavenging, lipid peroxidation prevention, antimicrobial, anticancer, antiviral, antihypertensive, antidiabetic, anti-inflammatory and anticoagulant properties have been found (Cheung, R. C. et al., Mar Drugs, 13, 4006, 2015, Suleria, H. A. R. et al., Trends in Food Science & Technology, 50:44, 2016). In addition, peptides isolated from various octopus species have been extensively studied due to their physiological activity against pathological conditions including inflammatory, autoimmune, hormone-related and cardiovascular diseases.

Inflammation is an evolutionarily conserved complex biological process that occurs in response to interruption of the tissue homeostasis caused by the presence of a biological, physical, or chemical stimulus. Under stress conditions, innate and adaptive immune systems coordinate to initiate controlled inflammatory responses to control tissue damages or pathogen attacks. However, uncontrolled or prolonged inflammatory responses are responsible for the pathogenesis of inflammatory diseases including cancers and tissue damage. During the last decade, the importance of Toll-like receptors (TLRs) during inflammatory responses has been reported. Specifically, several studies have identified overexpression of TLRs responsible for pathogenesis of chronic inflammatory diseases. According to the literature, there are 13 known TLRs found in mammalian cells. Among these 13 receptors, TLR2 and TLR4 that are expressed by macrophages in response to LPS were found to be associated with development of different inflammatory diseases via activation of the NF-KB pathway. Activation of NF-KB transcription factors then triggers gene transcription related to inflammatory mediators such as nitric oxide synthase (iNOS), cyclooxygenase (COX2), cytokines (IL1β, IL-6, and TNF-α), and chemokines (CCL3, CCL4, CCL5, and CXCL10). It has been reported that pro-inflammatory cytokines such as TNF-α, IL-1α, IL-1β, and IL-6 produced during the inflammatory responses contribute to the pathogenesis of diseases such as degeneration of the intervertebral discs, epilepsy, osteoarthritis, initiation and progression of cancer, and depression, and upregulate chemokine secretion from macrophages. Chemokines are small (7 to 13 kDa) heparin-binding proteins found to play an important role during acute and chronic inflammatory responses. Furthermore, chemokines such as CCL3 (macrophage inflammatory protein 1α, or MIP-1α), CCL4 (MIP-1β) CCL5 (RANTES), and CXCL10 produced during inflammatory responses primarily act as attractants for leukocytes (monocytes and neutrophils), and are regarded as mediators of chronic and acute inflammation. Recently, a number of studies reported that overexpression of chemokines is responsible for the pathogenesis of diseases such as osteoarthritis, liver diseases, and cancers. Additionally, overexpression of iNOS and COX2 in tissues leads to production of NO and PGE2, where those mediators have been demonstrated to play pivotal roles in the development of a number of inflammatory diseases.

Meanwhile, Octopus minor has a small body, short lifespan, and thin arms, compared to other octopus species, and is widely distributed along the coastal waters of East Asian countries such as Korea, Japan, and China. O. minor is an economically important sea food species and is considered a high nutrition and low-calorie food, and studies thereon have been conducted. However, most of the studies conducted on Octopus minor have been limited to genome studies, co-culture techniques, and nutritional analysis, and studies on its bioactive properties such as anti-inflammatory, anticancer and antioxidant properties have been limited.

Accordingly, the present inventors have made extensive efforts to find natural substances exhibiting anti-inflammatory effects from Octopus minor, and as a result, have found that, when macrophages activated by LPS are treated with a novel peptide derived from Octopus minor, the peptide exhibits an anti-inflammatory effect by inhibiting TLR-mediated NF-KB transcription, thereby completing the present invention.

### SUMMARY OF THE INVENTION

An object of the present invention is to provide a pharmaceutical composition for preventing or treating inflammatory disease containing, as an active ingredient, a natural peptide derived from Octopus minor.

In order to achieve the above object, the present invention provides a pharmaceutical composition for preventing or treating inflammatory disease containing, as an active ingredient, an antimicrobial peptide having five or more contiguous amino acid residues in the amino acid sequence of SEQ ID NO: 1:

| (SEQ ID NO: 1) | |
|---|---|
| | ¹GWLIRGAIHAGKAIHGLIHRRRH²³. |

The present invention also provides a method for preventing or treating an inflammatory disease comprising a step of administering the antimicrobial peptide.

The present invention also provides the use of the antimicrobial peptide for preventing or treating inflammatory disease.

The present invention also provides the use of the antimicrobial peptide for the manufacture of a therapeutic agent for inflammatory disease.

### BRIEF DESCRIPTION OF DRAWINGS

FIG. 1 shows predicted helical secondary and three-dimensional (3D) structures of the amino acid sequence of Octominin. (a) The helical wheel of Octominin shows the amino acid arrangement and the residue numbers which are counted from the amino (N) terminal of the peptide. The circles represent hydrophilic residues, the diamonds represent hydrophobic residues, and the pentagons represent positively charged residues. The most hydrophobic residue is green, and the amount of green decreases proportionally to the hydrophobicity, with zero hydrophobicity coded as yellow. (b) shows the three-dimensional structure of Octominin with positively charged residues.

FIG. 2 shows the results of examining the cyto-protective and NO inhibitory effect of Octominin in LPS-activated RAW 264.7 macrophages. (a): cytotoxicity of Octominin in macrophages, (b): NO production of Octominin in macrophages; (c): cytoprotective inhibitory effect of Octominin in LPS-stimulated macrophages, and (d): NO inhibitory effect of Octominin.

FIG. 3 shows the results of examining whether Octominin repress LPS-induced PGE2 and pro-inflammatory cytokine secretion from macrophages (PGE2 (a), IL6 (b), IL-113 (c), and TNF-α (d)). Experiments were triplicated, and the mean value is expressed as ±SD. Statistical significance was determined versus vehicle-treated control group (#<0.05 and ##<0.01) or LPS-treated group (*<0.05 and **<0.01).

FIG. 4 shows the results of qPCR performed to examine whether Octominin inhibits the production of pro-inflammatory cytokines and chemokines in LPS activated RAW 264.7 macrophages (IL-1β (a), IL-6 (b), TNF-α (c), CCL3 (d), CCL4 (e), CCL5 (f) and CXCL10 (g)). Experiments were triplicated to evaluate the data, and the mean value is expressed as ±SD. mRNA significance relative to the vehicle-treated control group was calculated using the Mann-Whitney U test (#<0.05 and ##<0.01) or LPS-treated group (*<0.05 and **<0.01).

FIG. 5 shows the results of examining whether Octominin inhibits iNOS and COX2 secretion from LPS-activated macrophages. (a) shows the relative mRNA expression level of iNOS measured using qPCR, and (b) shows the relative mRNA expression level of COX2 measured using qPCR. (c) shows the protein expression levels of iNOS and COX2 determined using Western blots, and (d) shows the results of analyzing the related expression of the bands of (c) using ImageJ software.

FIG. 6 shows the results of examining whether Octominin inhibits LPS-induced inflammation in macrophages via blocking TLR4/NFKB signal transduction. (a) shows the relative mRNA expression level of TLR2 measured using qPCR, and (b) shows the relative mRNA expression level of TLR4 measured using qPCR. (c) shows the results of Western blot analysis of cytoplasmic protein extracts, and (d) shows the results of Western blot analysis of nuclear protein extracts. Equal protein loading was controlled using antibodies against β-actin and nucleolin in cytosolic and nuclear extracts, respectively.

FIG. 7 shows molecular docking of Octominin to the crystal structure of the TLR4/MD-2 complex. (a): 2D diagram of Octominin; (b): the structure in which Octominin is docked to the crystal structure of the TLR4/MD-2 complex; and (c): amino acid binding sites of Octominin with TLR4/MD-2 complex.

FIG. 8 shows possible mechanisms of Octominin to inhibit LPS-activated inflammatory responses in RAW 264.7 macrophages.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

Peptides isolated from marine organisms may be used as promising therapeutic agents to treat various diseases, including antiviral, anticancer, antidiabetic, and antiobesity agents. Nonetheless, the anti-inflammatory effects and underlying molecular mechanisms of peptides isolated from octopus species such as Octopus minor are not well understood. In the present invention, it has been found that Octominin, a novel peptide derived from Octopus minor, can alleviate LPS-induced inflammation by down-regulating NO, PGE2, pro-inflammatory cytokines, and chemokines in macrophages. In addition it has been found that Octominin can reduce inflammatory responses in LPS-activated macrophages by blocking downstream activation of TLR/NF-kB signal transduction.

Therefore, in one aspect, the present invention is directed to a pharmaceutical composition for preventing or treating inflammatory disease containing, as an active ingredient, an antimicrobial peptide having five or more contiguous amino acid residues in the amino acid sequence of SEQ ID NO: 1:

| (SEQ ID NO: 1) | |
|---|---|
| | ¹GWLIRGAIHAGKAIHGLIHRRRH²³. |

In the present invention, the antimicrobial peptide may comprise a peptide having 5 or more, preferably 7 or more, more preferably 10 or more, most preferably 18 or more contiguous amino acid residues in the amino acid sequence of SEQ ID NO: 1, and may comprise an amino acid sequence in which one or more amino acid residues in a peptide having at least 7, more preferably at least 10, most preferably at least 18 contiguous amino acid residues are conservatively substituted. Conservative amino acid substitution may involve substitution of a native amino acid residue with a non-native residue such that there is little or no effect on the size, polarity, charge, hydrophobicity, or hydrophilicity of the amino acid residue at that position.

The antimicrobial peptide according to the present invention may have the amino acid sequence of SEQ ID NO: 1, or an amino acid sequence having at least 90%, preferably at least 95%, more preferably at least 98% homology to 5 or more, preferably 7 or more, more preferably 10 or more, most preferably 18 or more contiguous amino acid residues in the amino acid sequence of SEQ ID NO: 1.

Preferably, the antimicrobial peptide according to the present invention may comprise the amino acid sequence of SEQ ID NO: 2, and may comprise 18 to 23 contiguous amino acid residues. More preferably, the antimicrobial peptide may be represented by any one of the amino acid sequences of SEQ ID NO: 1 to SEQ ID NO: 6:

| (SEQ ID NO: 1) | |
|---|---|
| | ¹GWLIRGAIHAGKAIHGLIHRRRH²³ |
| (SEQ ID NO: 2) | |
| | ¹GWLIRGAIHAGKAIHGLI¹⁸ |
| (SEQ ID NO: 3) | |
| | ¹GWLIRGAIHAGKAIHGLIH¹⁹ |
| (SEQ ID NO: 4) | |
| | ¹GWLIRGAIHAGKAIHGLIHR²⁰ |
| (SEQ ID NO: 5) | |
| | ¹GWLIRGAIHAGKAIHGLIHRR²¹ |
| (SEQ ID NO: 6) | |
| | ¹GWLIRGAIHAGKAIHGLIHRRR²² |

Preferably, the antimicrobial peptide according to the present invention may comprise the amino acid sequence of SEQ ID NO: 2, and may further comprise, at the C-terminus of SEQ ID NO: 2, 1 to 5 contiguous amino acid residues among the amino acid sequence of SEQ ID NO: 7.

| (SEQ ID NO: 7) | |
|---|---|
| | “HRRRH” |

As a result of analyzing the amino acid sequence of the antimicrobial peptide Octominin having the sequence of SEQ ID NO: 1 according to the present invention, Octominin showed the following characteristics: total net charge: +5, total hydrophobic residue ratio: 43%, and Boman index: 1.86 kcal/mol. The secondary structure of Octominin was predicted to have an alpha helix and a beta sheet. The molecular weight of the synthesized Octominin was 2,625.2 Da, and the purity thereof was 92.5%.

Macrophages play a critical role during inflammatory responses, and activated macrophages secrete NO, PGE2 and pro-inflammatory mediators. In response to LPS, macrophages produce NO and PGE2 through overexpression of iNOS and COX2, respectively. Excessive production of NO and PGE2 plays an important role in the pathogenesis of various inflammatory diseases. Thus, alternative compounds that inhibit/downregulate the expression of inflammatory mediators such as iNOS and COX2 can help in the treatment of inflammatory diseases, and thus can be developed as therapeutic agents for inflammatory diseases.

TLR4 is an important member receptor of TLRs for identifying iLPS and is responsible for initiating LPS-mediated inflammatory responses. MD-2 and TLR4 form a heterodimer (TLR4/MD-2) that identifies a common pattern in LPS. LPS binding leads to the formation of an m-type receptor multimer consisting of two copies of the symmetrically arranged TLR4/MD-2/LPS complex. Generally, LPS interacts with the hydrophobic pocket in MD-2 and directly links the two components of the TLR4/MD-2/LPS multimer. Lipid chains of LPS (five chains) are submerged in a pocket located in MD-2 and another chain is exposed to the surface of MD-2, forming a hydrophobic interaction with the conserved phenylalanine of TLR4. Therefore, it is essential for LPS to bind with TLR4/MD-2 heterodimer to trigger inflammatory responses.

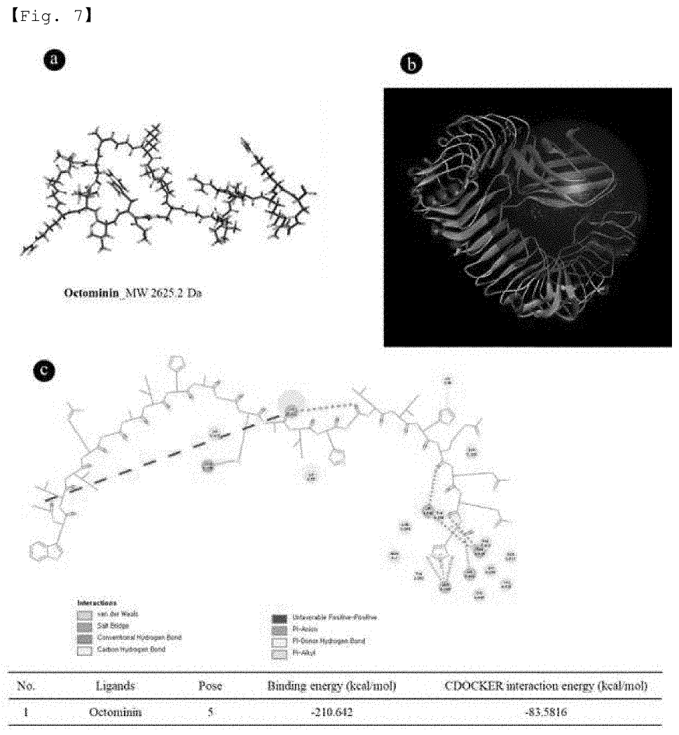

In one example of the present invention, the ability of Octominin to bind to the TLR4/MD-2 complex was evaluated in silico, and Octominin has a potential to act as an anti-inflammatory agent in LPS-activated macrophages (see Example and FIG. 7). According to in silico docking results, Octominin was buried in the pocket of the TLR4/MD-2 complex by interacting with amino acid sites located in the TLR4/MD-2 heterodimer. This steric hindrance could weaken or reduce the binding of LPS with TLR4/MD-2.

In the present invention, a series of in vitro experiments were conducted to evaluate the anti-inflammatory properties of Octominin.

In another example of the present invention, it was confirmed that Octominin effectively inhibited NO and PGE2 secretion from activated macrophages by down-regulating mRNA/protein expression of iNOS and COX2 without reducing cell viability.

In another example of the present invention, it was confirmed that Octominin significantly inhibited LPS-induced mRNA expression and protein expression of TNF-α, IL-113 and IL-6 in a dose-dependent manner.

Synthesized peptides and antimicrobial peptides (AMPS) have been found to exhibit similar anti-inflammatory properties in LPS-activated macrophages. For instance, synthesized peptides and AMPS were found to inhibit LPS-induced inflammation in RAW 264.7 macrophages through inhibition of iNOS, COX2, and pro-inflammatory cytokine secretion. Thus, examination was made as to whether Octominin would exert anti-inflammatory effects in LPS-activated macrophages by repressing pro-inflammatory mediators.

In another example of the present invention, it was confirmed that Octominin could repress LPS-induced chemokine production from LPS-activated RAW 264.7 macrophages including CCL3, CCL4, CCL5 and CXCL10. Chemokines are a group of small proteins secreted by macrophages in response to pro-inflammatory cytokines and they play an important role during the inflammatory responses. In general, chemokines modulate different activities of leukocytes during inflammatory responses such as leucocyte activation, chemotaxis, and recruitment of activated macrophages cell trafficking, cell proliferation, and direction of neutrophils and T cells toward the cite of inflammation. Hence, therapeutic compounds targeting chemokine secretion could contribute to inhibiting this inflammatory process.

In another example of the present invention, it was confirmed that the chemokine secretions from activated macrophages treated with Octominin were decreased similar to the pro-inflammatory cytokine production from activated macrophages. These results suggest that Octominin is capable of inhibiting LPS-activated inflammatory responses, as well as the development of inflammatory symptoms by reducing leucocyte and neutrophil recruitment to infected areas.

Several studies have demonstrated that exposure of macrophages to LPS causes prompt cellular responses. Upon exposure of macrophages to LPS, MyD88 is activated by pattern receptors such as TLRs. Subsequently, the activated MyD88 activates the inhibitor of the kappa B kinase (IKK) complex which is capable of inducing phosphorylation of the inhibitor of kappa B (IKB). The phosphorylation of IKB liberates cytoplasmic NF-KB dimers (p50 and p65) and allows translocation thereof into the nucleus. Subsequently, translocation of NF-KB facilitates the transcription of genes associated with inflammatory responses in macrophages. Previously, several studies reported that peptides separated from different organisms are capable of inhibiting inflammatory cytokine and chemokine secretion by alleviating NF-KB binding to the nucleus (Glaeser, J. D. et al., Tissue Eng Part A 24:1641, 2018).

In another example of the present invention, it was confirmed that phosphorylation of p50 and p65 and their nucleus translocations were significantly inhibited by pretreatment with Octominin. As mentioned above, the activation of NF-KB is triggered by LPS after binding to TLRs. The TLR-mediated signaling pathways regulate the release of pro-inflammatory cytokines, as well as chemokines, and are identified as one of the novel therapeutic targets for inflammatory diseases.

Thus, in another example of the present invention, gene expression levels of TLR2 and TLR4 were assessed using RT-qPCR. Similar to the previous studies performed to evaluate anti-inflammatory mechanisms of different peptides, the present inventors have also found that Octominin has a potential to repress LPS-activated TLRs mRNA expression from LPS-challenged macrophages.

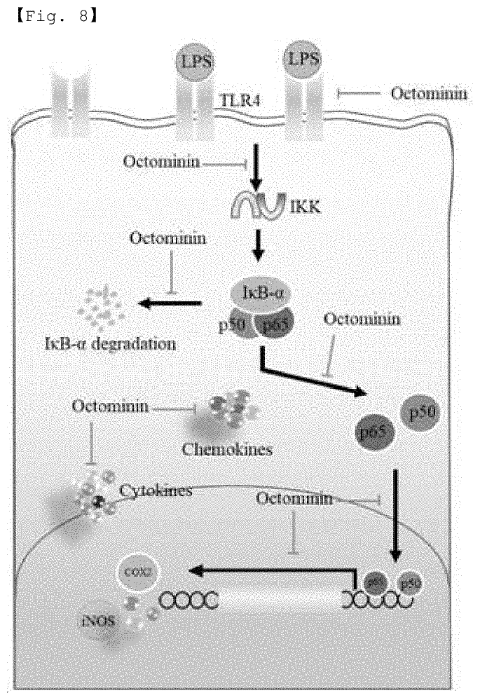

Thus, the results of the present invention suggest that the anti-inflammatory activity of Octominin is partially mediated through inhibiting NF-KB binding to the nucleus via suppressing TLRs. A possible mechanism of action by which Octominin modulates LPS-activated inflammatory responses in RAW 264.7 macrophages is shown in FIG. 8.

In the present invention, examples of inflammatory diseases include, but are not limited to, atopy, psoriasis, arthritis, dermatitis, allergy, osteoarthritis, rhinitis, otitis media, sore throat, tonsillitis, periodontitis, gingivitis, inflammatory eye disease, cystitis, nephritis, rheumatoid arthritis, spondylitis, inflammatory bowel disease, hepatitis, sepsis, alcoholic liver disease, non-alcoholic fatty liver, various cancers, epilepsy, intervertebral disc degeneration, and the like.

The pharmaceutical composition of the present invention is preferably administered orally or parenterally.

For oral administration, the pharmaceutical composition of the present invention may be orally administered in any orally acceptable dosage form including, but not limited to, pills, dragées, capsules, liquids, gels, syrups, slurries, and suspensions.

In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient may be combined with emulsifying and suspending agents. If desired, certain sweetening, flavoring and/or coloring agents may be added.

The pharmaceutical composition for oral administration may be prepared by mixing the active ingredient with a solid excipient, and may be prepared in the form of granules for preparation in the form of tablets or dragées.

Suitable excipients include sugar forms such as lactose, sucrose, mannitol and sorbitol, starch from corn, flour, rice, potatoes or other plants, or celluloses such as methyl cellulose, hydroxypropylmethyl-cellulose or sodium carboxymethylcellulose, carbohydrates such as gums, including Arabic gum and tragacanth gum, or protein fillers such as gelatin and collagen. If necessary, a disintegrant or solubilizer, such as crosslinked polyvinylpyrrolidone, agar and alginic acid or a salt form thereof, for example, sodium alginate, may be added.

As used herein, the term “parenteral” includes subcutaneous, transdermal, intravenous, intramuscular, intra-articular, intra-synovial, intrasternal, intradural, intra-lesional and intra-cranial injection or infusion techniques.

The pharmaceutical composition of the present invention may be in the form of a sterile injectable formulation as a sterile injectable aqueous or oleaginous suspension. This suspension may be formulated according to techniques known in the art using suitable dispersing or wetting agents (e.g., Tween 80), and suspending agents. The sterile injectable preparation may also be a sterile injectable solution or suspension in a non-toxic parenterally acceptable diluent or solvent, for example, a solution in 1,3-butanediol. Other examples of acceptable vehicles and solvents that may be employed include mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions.

In a preferred embodiment, for parenteral administration, the pharmaceutical composition of the present invention may be prepared as an aqueous solution. Preferably, a physically appropriate buffer, such as Hank's solution, Ringer's solution, or physically buffered saline, may be used. Water-soluble injectable suspensions may include a substrate that can increase the viscosity of the suspension, such as sodium carboxymethyl cellulose, sorbitol, or dextran. Additionally, suspensions of the active ingredient may be prepared as oily injection suspensions. Suitable lipophilic solvents or vehicles include fatty oils such as sesame oil, or synthetic fatty acid esters, such as ethyl oleate or triglycerides, or liposomes.

Polycationic amino polymers may also be used as carriers. The suspension may optionally contain suitable stabilizers or agents to increase the solubility of the compound and to allow for more concentrated solutions.

The pharmaceutical composition of the present invention may also be administered in the form of a suppository for rectal administration. This composition may be prepared by mixing the compound of the present invention with a suitable non-irritating excipient that is solid at room temperature but liquid at rectal temperature. Such materials include, but are not limited to, cocoa butter, beeswax and polyethylene glycols.

When the pharmaceutical composition of the present invention is to be applied topically to the skin, the pharmaceutical composition may be formulated in a suitable ointment containing the active ingredient suspended or dissolved in one or more carriers. Carriers for topical administration of the compound of the present invention include, but are not limited to, mineral oil, liquid paraffin, white petrolatum, propylene glycol, polyoxyethylene, polyoxypropylene, emulsifying wax, and water. Alternatively, the pharmaceutical compositions may be formulated into a suitable lotion or cream containing the active ingredient suspended or dissolved in one or more carriers. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical composition of the present invention may also be topically applied to the lower intestinal tract in a rectal suppository formulation or in a suitable enema formulation. Topically-transdermal patches are also included in the present invention.

The pharmaceutical composition of the present invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

It will be appreciated that a specific effective amount for a specific patient may vary depending on several factors, including the activity of the specific compound used, the patient's age, weight, general health, sex, diet, administration time, route of administration, excretion rate, drug formulation, and the severity of the specific disease to be prevented or treated.

In another aspect, the present invention is directed to a method for preventing or treating an inflammatory disease comprising a step of administering the antimicrobial peptide.

In another aspect, the present invention is directed to the use of the antimicrobial peptide for preventing or treating inflammatory disease.

In another aspect, the present invention is directed to the use of the antimicrobial peptide for the manufacture of a therapeutic agent for inflammatory disease.

Hereinafter, the present invention will be described in more detail with reference to examples. These examples are only for illustrating the present invention in more detail, and it will be obvious to those of ordinary skill in the art that the scope of the present invention is not to be construed as being limited by these examples.

### Example 1: Synthesis and Physicochemical Properties of Octominin

Healthy Octopus minor with a mean body weight of about 120 g were obtained from Shinan mudflat on the southwest coast of Korea. Prior to the experiments, Octopus minor were acclimatized over a week in a 300 L flow-through system tank under the conditions of salinity 34±1 psu and pH 8.0±0.4 at 20±1° C.

Transcriptome Sequencing of Octopus minor

Total RNA was extracted from 13 tissues (eye, brain, branchial heart, liver, buccal mass, heart, kidney, ovary, siphon, poison gland, skin, and suckers) parts of O. minor using the RNeasy Mini Kit (Qiagen, Hilden, Germany). The purity of the extracted RNA was confirmed using an Agilent Bioanalyzer. Isoform sequencing was performed using pooled RNA collected from 13 organs. Library construction and sequencing were performed using PacBio RS II (DNA Link, Inc., Seoul, Korea).

Design and Synthesis of Defense Peptide Octominin Derived from Octopus minor

A defense protein gene was screened by screening the transcriptome sequence obtained from the Octopus minor transcriptome database. On the basis of the N-terminal amino acid sequence of the defense protein, a novel peptide consisting of 23 amino acid residues (¹GWLIRGAIHAGKAIHGLIHRRRH²³) was designed and named Octominin. Octominin was synthesized using solid-phase peptide synthesis technology (AnyGen Co., Korea), and purified by reverse-phase HPLC using a C18 analytical column (SHIMADZU C18, Shimadzu HPLC Lab Solution, Japan). The molecular weight of Octominin was confirmed using mass spectrometry (AXIMA Confidence, MALDI-TOF, Shimadzu).

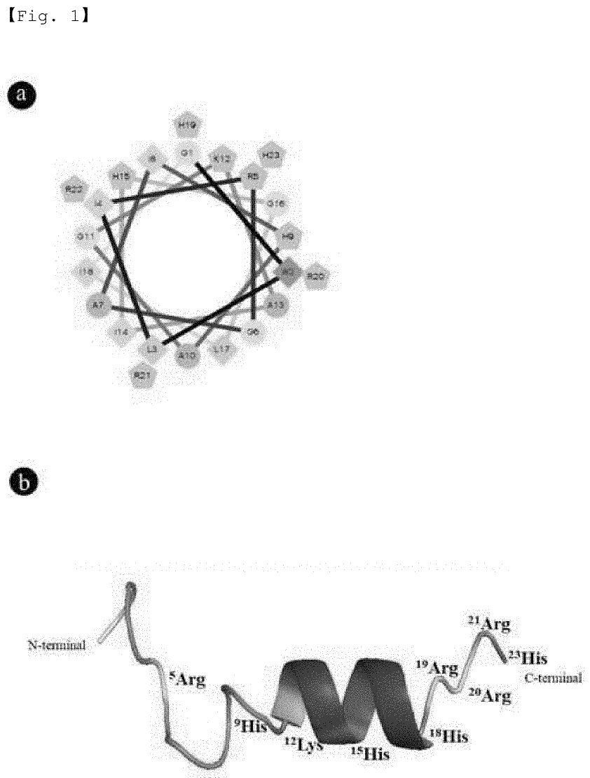

Based on the peptide prediction results, Octominin had a total net charge of +5, high number of positively charged residues (Lys-K, Arg-R and His-H), and hydrophobic residues (Ile-1, Leu-L, and Ala-Trp-W) with a total hydrophobic ratio of 43% and a Boman index (protein binding potential) of 1.86 kcal/mol. Negatively charged amino acids such as aspartate and glutamate were absent in the Octominin sequence. There are a total of 8 hydrophobic residues on the same surface of the peptide and alpha helices. The secondary and three-dimensional structures of Octominin are shown in FIG. 1. As a result of analysis of the secondary structure of Octominin, alpha helices and beta sheets were identified as expected. The molecular weight of the synthetic Octominin was 2626.1 Da and the purity thereof was 92.5%.

### Example 2: Examination of Cytotoxic Effect of Octominin in LPS-Activated Macrophages and NO Secretion

As macrophages, RAW 264.7 cells (American Type Culture Collection; ATCCUSA) were cultured in DMEM (Gibco/BRL, Canada) supplemented with 10% heat inactivated FBS and 1% antibiotics in 37° C. at 100% humidity under 5% CO₂.

RAW 264.7 cells were cultured in 24-cell culture plates (45,000 cells/well) and treated with LPS (1 μg/mL) alone or in combination with Octominin (62.5, 125, and 250 and 500 μg/mL) in a total volume of 500 μL. After 24 hours, culture medium containing 3-(4,5-dimethylthiazol-2-yl)-2,5-diphenyltetrazolium bromide (MTT) (0.1 mg/mL) was added to each well. After 1 hour, culture medium was removed, and formazan crystals were dissolved in dimethyl sulfoxide (DMSO) and measured at 550 nm using a microplate reader. The concentration of NO in the culture medium was determined by Griess reagent (Abekura et al. Int Immunopharmacol 68:156, 2019). The cell viability of the control group untreated with Octominin was designated as 100%, which indicates no cytotoxicity

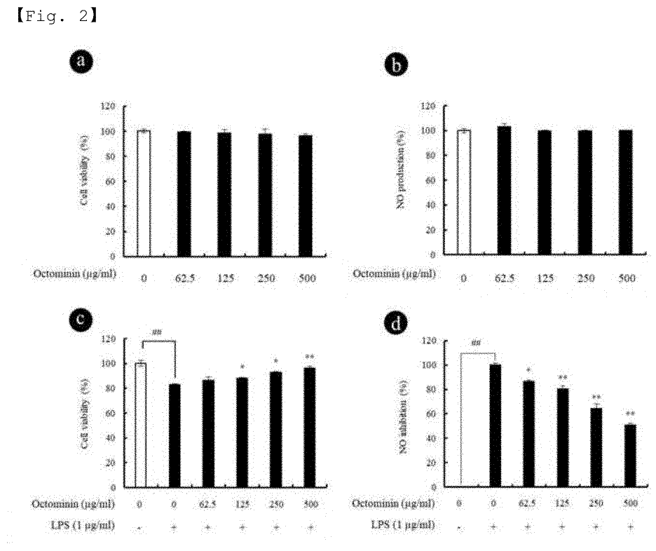

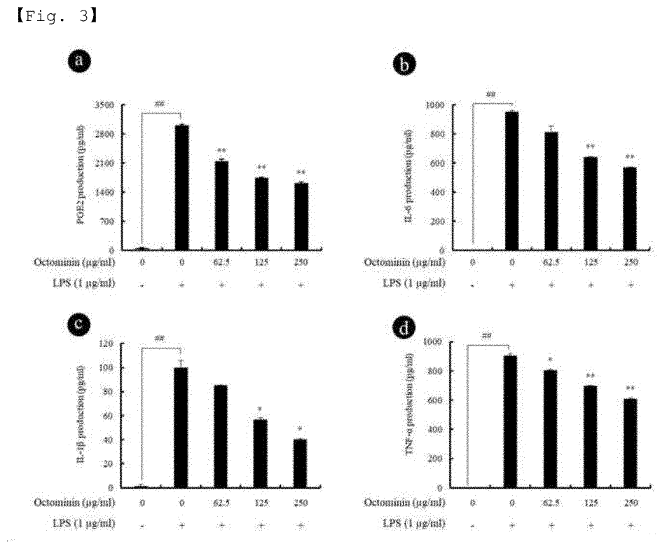

As a result, as shown in FIG. 2, the concentrations of Octominin between 62.5 and 500 μg/mL did not show any significant cell viability reduction (FIG. 3a) or NO production (FIG. 3b) compared with the control group after hours of incubation with RAW 264.7 macrophages. In addition, the protective effects of Octominin on macrophages were evaluated, and values between 62.5 and 500 μg/mL were considered non-toxic to macrophages. Octominin showed enhanced cyto-protective effect and inhibited NO production in a dose-dependent manner from 62.5 to 500 μg/mL of Octominin in LPS-activated macrophages (FIGS. 3c and 3d). Considering amounts required for biological assays, cyto-protective data, and NO inhibition data, the present inventors decided to use 62.5, 125, and 250 μg/mL concentrations of Octominin in subsequent studies.

### Example 3: Examination of the Ability of Octominin to Inhibit LPS-Induced PGE2 and Pro-Inflammatory Cytokine Secretion

Whether Octominin inhibits LPS-induced PGE2 and pro-inflammatory cytokine secretion from RAW 264.7 macrophages was examined using ELISA.

Macrophages were exposed to various concentrations of Octominin for 1 hour prior to activation with LPS. After culturing the cells for 24 hours, the cell-free culture supernatant was collected. The level of PGE2 in the culture supernatant was analyzed using an ELISA kit (R & D System Inc., USA), and the levels of TNF-α, IL-1β and IL-6 were analyzed using an ELISA kit (BD Biosciences; San Jose, Calif., USA).

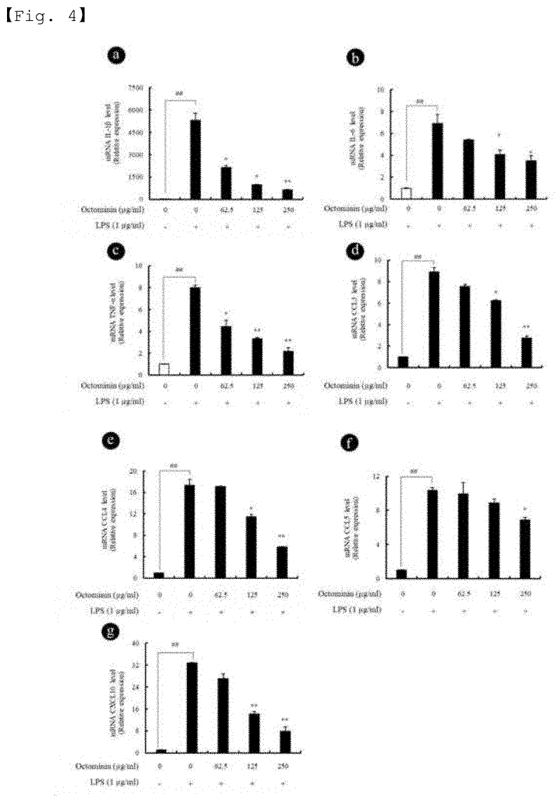

As a result, as shown in FIG. 3, LPS stimulation significantly increased the levels of PGE2, IL-1β, IL-6, and TNFα in the culture supernatants of RAW 264.7 cells after 24 hours. However, treatment with Octominin (62.5 to 250 μg/mL) prior to LPS activation significantly inhibited PGE2 and pro-inflammatory cytokines (IL-6 FIG. 4b, IL-1β FIG. 4c, and TNF-α FIG. 3d) compared with the LPS-activated group.

### Example 4: Examination of Inhibitory Effects of Octominin Against Gene Expression of Chemokines and Pro-Inflammatory Cytokine in Activated Macrophages

In order to examine the effect of Octominin on the transcription of pro-inflammatory cytokine and chemokine signaling genes, qPCR analysis of IL-1β, IL-6, TNF-α, CCL3, CCL4, CCL5, and CXCL10 in LPS-activated macrophages was performed.

RAW 264.7 cells (150,000 cells/well) were plated into 6-well plates and pretreated with different concentrations (62.5, 125, and 250 μg/mL) of Octominin, then stimulated with 1 μg/mL LPS for 6 hours. RAW 264.7 cells were collected and total RNA was isolated using the Trizol regent according to the manufacturer's instructions. The quantity of total RNA was determined at 260 and 280 nm. Total RNA (5 μg) was reverse-transcribed and cDNA was synthesized using a cDNA synthesis kit (TaKaRa, Tokyo, Japan). Target genes were then amplified by RT-PCR using oligo dT primers (Bioneer, Korea). The gene expression levels relative to GAPDH used as an internal control were analyzed by the 2^{-ΔΔCT} method. The data are expressed as the mean±standard error (SE) of the relative mRNA expression level from three consecutive studies. The Mann-Whitney U test was used to determine the significance of gene expression levels. The primers used in the present invention are shown in Table 1 below.

TABLE 1

| | | | SEQ ID |
|---|---|---|---|
| Gene | Primer | Sequence (5′ → 3′) | NO |
| GAPDH | Sense | AAGGGTCATCATCTCTGCCC | 7 |
| | Antisense | GTGATGGCATGGACTGTGGT | 8 |
| iNOS | Sense | ATGTCCGAAGCAAACATCAC | 9 |
| | Antisense | TAATGTCCAGGAAGTAGGTG | 10 |
| COX2 | Sense | CAGCAAATCCTTGCTGTTCC | 11 |
| | Antisense | TGGGCAAAGAATGCAAACAT | 12 |
| | | C | |
| IL-1β | Sense | CAGGATGAGGACATGAGCAC | 13 |
| | | C | |
| | Antisense | CTCTGCAGACTCAAACTCCA | 14 |
| | | C | |
| IL-6 | Sense | GTACTCCAGAAGACCAGAGG | 15 |
| | Antisense | TGCTGGTGACAACCACGGCC | 16 |
| TNF-α | Sense | TTGACCTCAGCGCTGAGTTG | 17 |
| | Antisense | CCTGTAGCCCACGTCGTAGC | 18 |
| TLR2 | Sense | CAGCTGGAGAACTCTGACCC | 19 |
| | Antisense | CAAAGAGCCTGAAGTGGGAG | 20 |
| TLR4 | Sense | CAACATCATCCAGGAAGGC | 21 |
| | Antisense | GAAGGCGATACAATTCCACC | 22 |
| CCL3 | Sense | CGGAAGATTCCACGCCAATT | 23 |
| | | CATCG | |
| | Antisense | CAGATCTGCCGGTTTCTCTT | 24 |
| | | AGTCAGG | |
| CCL4/ | Sense | CAGCTCTGTGCAAACCTAAC | 25 |
| MIP-1 | | CC | |
| | Antisense | AACCCTGGAGCACAGAAGGC | 26 |
| CCL5/ | Sense | TGTTTGTCACTCGAAGGAAC | 27 |
| RANTES | | CG | |
| | Antisense | TGGGGGTCAGAATCAAGAAA | 28 |
| | | CCC | |
| CXCL10 | Sense | ATGACGGGCCAGTGAGAATG | 29 |
| | | AGG | |
| | Antisense | GCACTGCACAAGAAGATGCG | 30 |

As a result, as shown in FIG. 4, it was confirmed that LPS remarkably promoted gene transcription of IL-1β (FIG. 4a), IL-6 (FIG. 4b), and TNF-α (FIG. 4c) compared with the vehicle-treated control group.

However, Octominin significantly and dose-dependently repressed the elevated cytokine levels observed in LPS-activated macrophages. In addition, as shown in FIGS. 4d to 4g, the expression of chemokines including CCL3 (MIP-1α) CCL4 (MIP-1β), CCL-5 (RANTES), and CXCL10 was significantly elevated after LPS stimulation, indicating that treatment with Octominin significantly and dose-dependently repressed the elevated expression of these chemokines.

### Example 5: Examination as to Whether Octominin Represses LPS-Induced iNOS and COX2 Activity

Examination was made as to whether LPS-induced iNOS and COX2 activity in LPS-activated RAW 264.7 macrophages was repressed by Octominin treatment.

The expression of key enzymes responsible for the NO and PGE2 production, including iNOS and COX2, respectively, was examined. qPCR was performed in the same manner as in Example 4 using the primers shown in Table 1 above, and the relative mRNA expression levels were calculated using the 2-ΔΔCt method.

For Western blotting, nuclear and cytoplasmic proteins were extracted using a nuclear and cytoplasmic protein extraction kit (Thermo Scientific; Rockford, USA). An equal amount of protein was electrophoresed in 10% SDS-PAGE, and the separated proteins were transferred onto PVDF membranes. Then, PVDF membranes were incubated with primary antibody and then incubated with HRP-conjugated secondary antibody. The protein bands on PVDF membranes were detected by using an enhanced chemiluminescent substrate, and membranes were captured using a FUSION SOLO Vilber Lourmat system. Signal intensities of protein bands were determined by densitometry using ImageJ (version 1.4).

Primary and secondary antibodies against iNOS, COX2 and β-actin were purchased from Santa Cruz Biotechnology (USA) and used.

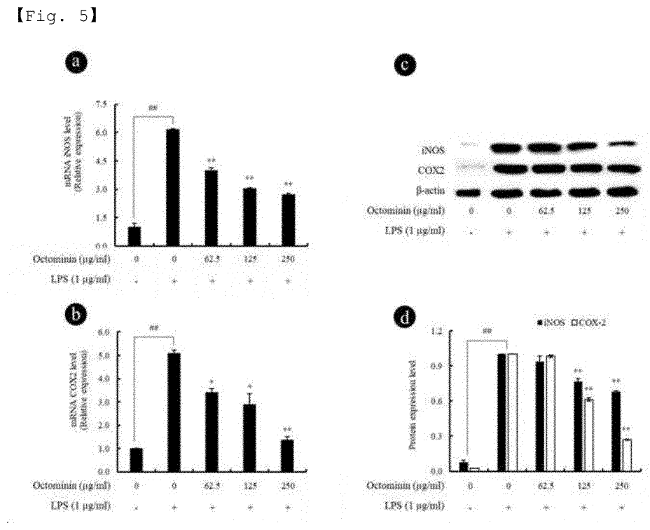

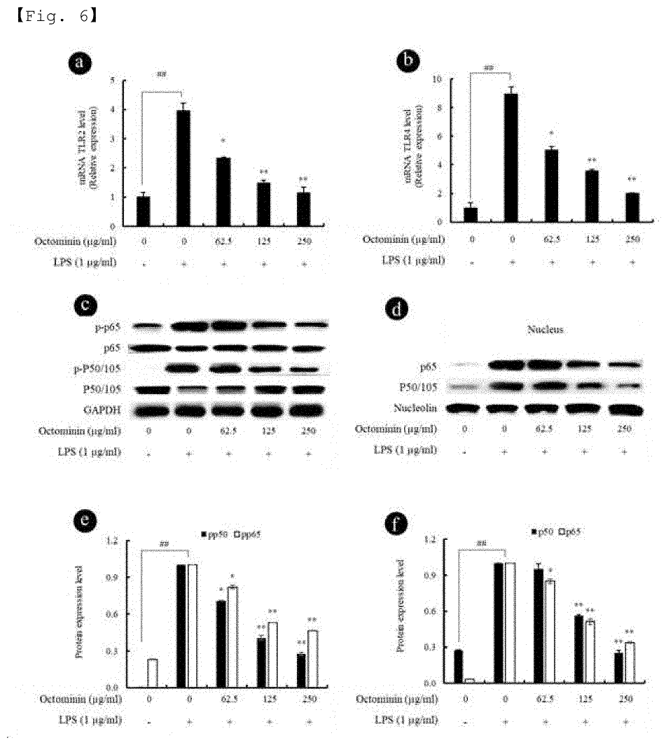

As a result, as shown in FIGS. 5a and 5b, the mRNA expression levels of iNOS and COX2 were significantly inhibited by Octominin dose-dependently, from activated RAW 264.7 cells. Similar to the mRNA expression results, LPS-stimulated RAW 264.7 cells had upregulated protein levels of iNOS and COX2 (FIGS. 6c and 6d), whereas this effect was significantly and dose-dependently inhibited by Octominin (62.5 to 250 μg/mL).

### Example 6: Examination of the Ability of Octominin to Represses Expression Levels of TLRs and NF-KB Phosphorylation in LPS-Activated Macrophages

It is a well-known fact that LPS activates Toll-like receptors (TLRs) and leads to the activation of NF-KB through the activation of the upstream proteins. Thus, in this Example, protein expression levels of NF-KB were examined using Western blots, and the mRNA expression levels of TLR2 and TLR4 were examined using qPCR.

Nuclear and cytoplasmic proteins were extracted using a nuclear and cytoplasmic protein extraction kit (Thermo Scientific; Rockford, USA). An equal amount of protein was electrophoresed in 10% SDS-PAGE, and the separated proteins were transferred onto PVDF membranes. Then, PVDF membranes were incubated with primary antibody and then incubated with HRP-conjugated secondary antibody. The protein bands on PVDF membranes were detected by using an enhanced chemiluminescent substrate, and membranes were captured using a FUSION SOLO Vilber Lourmat system. Signal intensities of protein bands were determined by densitometry using ImageJ (version 1.4).

Primary and secondary antibodies against phospo-P50, P50, phospo-P65, P65, nucleolin, and β-actin were purchased from Santa Cruz Biotechnology (USA) and used.

As a result, as shown in FIG. 6, the gene expression levels of TLR2 (FIG. 6a) and TLR4 (FIG. 6b) were significantly upregulated by the LPS treatment as expected. However, Octominin treatment downregulated the elevated TLRs gene expression levels in LPS-activated macrophages. In addition, as shown in FIGS. 6c and 6e, the phosphorylation of NF-KB subunit p50 and p65 in the cytosol was significantly increased after LPS treatment, whereas Octominin significantly downregulated the levels of phosphorylated p50 and p65 in cytosol, suggesting that Octominin has a potential to inhibit NF-KB in cytosol. As a result of Western bolt analysis, as shown in FIG. 6d, nucleus translocation levels of NF-KB, p50 and p65 in LPS-activated macrophages were evaluated, and expression levels of p50 and p65 in LPS-activated macrophages were dose-dependently downregulated by Octominin treatment. However, 62.5 μg/mL of Octominin did not show any significant p50 inhibition in nuclear protein extract (FIG. 6f).

### Example 7: Examination of Inhibitory Effect of Octominin on TLR4/MD-2 Complex Formation in Silico

In order to further confirm the anti-inflammatory effect of Octominin observed in LPS-activated macrophages (FIG. 7a), a design for predicting the ability of Octominin to bind to the TLR4/MD-2 complex was performed.

The crystallographic structure of TLR4/myeloid differentiation factor 2 (MD-2) (PDB: 3FXI) was obtained from the Protein Data Bank (PDB) (http://www.rcsb.org/pdb). The two-dimensional structure of Octominin was drawn by MDL ISIS Draw 2.5 standalone software and converted into three-dimensional structures using the Accelrys Discovery Studio 3.0 (Accelrys, Inc.). Binding was evaluated based on CDOCKER.

To explore the possible binding modes, Octominin was virtually docked in a 3D model of the TLR4/MD-2 complex using Accelrys Discovery Studio 3.0 (Accelrys, Inc.) (FIG. 7b). As shown in FIG. 7b, molecular docking showed that Octominin moved into the pocket of the TLR4/MD-2 complex and interacted with several amino acids sites such as SER120, SER 917, LEU293, LYS941, and THR919 (see FIG. 7C) in TLR4 which occupied the space and weakened the binding of LPS with TLR4/MD-2.

### INDUSTRIAL APPLICABILITY

The novel antibacterial peptide or fragment thereof according to the present invention may efficiently inhibit pro-inflammatory cytokines and chemokines responsible for inflammatory responses in LPS-activated macrophages, and thus may be effectively used to prevent or treat inflammatory disease.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

The present invention was made with the support of the national support project as follows.

[Project number] 2019R1A2C1087028

[Name of Ministry] Ministry of Science and ICT

[Research and Management Institution] National Research Foundation of Korea

[Research project name] Mid-level researcher support project

[Project title] Development of nano drug delivery system based on target-selective fusion biodefense peptide derived from Octopus minor

[Name of institution performing the project] National Marine Biodiversity Institute of Korea

[Research period] Sep. 1, 2019 through Feb. 28, 2023

### Sequence List Free Text

Electronic file is attached

1. A method for preventing or treating inflammatory disease comprising administering a pharmaceutical composition containing, as an active ingredient, an antimicrobial peptide having five or more contiguous amino acid residues in the amino acid sequence of SEQ ID NO: 1:

| (SEQ ID NO: 1) | |
|---|---|
| | ¹GWLIRGAIHAGKAIHGLIHRRRH²³ |

to a subject in need thereof.

2. The method of claim 1, wherein the antimicrobial peptide comprises the amino acid sequence of SEQ ID NO: 2, and comprises 18 to 23 contiguous amino acid residues:

| (SEQ ID NO: 2) | |
|---|---|
| | ¹GWLIRGAIHAGKAIHGLI¹⁸ |

3. The method of claim 2, wherein the antimicrobial peptide comprises any one amino acid sequence selected from among SEQ ID NO: 1 to SEQ ID NO: 6.

4. The method of any one of claims 1 to 3, wherein the inflammatory disease is selected from the group consisting of atopy, psoriasis, arthritis, dermatitis, allergy, osteoarthritis, rhinitis, otitis media, sore throat, tonsillitis, periodontitis, gingivitis, inflammatory eye disease, cystitis, nephritis, rheumatoid arthritis, spondylitis, inflammatory bowel disease, hepatitis, sepsis, alcoholic liver disease, non-alcoholic fatty liver, various cancers, epilepsy, and disc degeneration.


SEQUENCE LISTING (31 sequences)

SEQ ID NO: 1 - protein, 23 aa, Artificial Sequence
  octominin
GWLIRGAIHAGKAIHGLIHRRRH

SEQ ID NO: 2 - protein, 18 aa, Artificial Sequence
  antibacterial peptide
GWLIRGAIHAGKAIHGLI

SEQ ID NO: 3 - protein, 19 aa, Artificial Sequence
  antibacterial peptide
GWLIRGAIHAGKAIHGLIH

SEQ ID NO: 4 - protein, 20 aa, Artificial Sequence
  antibacterial peptide
GWLIRGAIHAGKAIHGLIHR

SEQ ID NO: 5 - protein, 21 aa, Artificial Sequence
  antibacterial peptide
GWLIRGAIHAGKAIHGLIHRR

SEQ ID NO: 6 - protein, 22 aa, Artificial Sequence
  antibacterial peptide
GWLIRGAIHAGKAIHGLIHRRR

SEQ ID NO: 7 - protein, 5 aa, Artificial Sequence
  partial sequence
HRRRH

SEQ ID NO: 8 - DNA, 20 bp, Artificial Sequence
  GAPDH sense primer
aagggtcatcatctctgccc

SEQ ID NO: 9 - DNA, 20 bp, Artificial Sequence
  GAPDH antisense primer
gtgatggcatggactgtggt

SEQ ID NO: 10 - DNA, 20 bp, Artificial Sequence
  iNOS sense primer
atgtccgaagcaaacatcac

SEQ ID NO: 11 - DNA, 20 bp, Artificial Sequence
  iNOS antisense primer
taatgtccaggaagtaggtg

SEQ ID NO: 12 - DNA, 20 bp, Artificial Sequence
  COX2 sense primer
cagcaaatccttgctgttcc

SEQ ID NO: 13 - DNA, 21 bp, Artificial Sequence
  COX2 antisense primer
tgggcaaagaatgcaaacatc

SEQ ID NO: 14 - DNA, 21 bp, Artificial Sequence
  IL-1beta sense primer
caggatgaggacatgagcacc

SEQ ID NO: 15 - DNA, 21 bp, Artificial Sequence
  IL-1beta antisense primer
ctctgcagactcaaactccac

SEQ ID NO: 16 - DNA, 20 bp, Artificial Sequence
  IL-6 sense primer
gtactccagaagaccagagg

SEQ ID NO: 17 - DNA, 20 bp, Artificial Sequence
  IL-6 antisense primer
tgctggtgacaaccacggcc

SEQ ID NO: 18 - DNA, 20 bp, Artificial Sequence
  TNF-alpha sense primer
ttgacctcagcgctgagttg

SEQ ID NO: 19 - DNA, 20 bp, Artificial Sequence
  TNF-alpha antisense primer
cctgtagcccacgtcgtagc

SEQ ID NO: 20 - DNA, 20 bp, Artificial Sequence
  TLR2 sense primer
cagctggagaactctgaccc

SEQ ID NO: 21 - DNA, 20 bp, Artificial Sequence
  TLR2 antisense primer
caaagagcctgaagtgggag

SEQ ID NO: 22 - DNA, 19 bp, Artificial Sequence
  TLR4 sense primer
caacatcatccaggaaggc

SEQ ID NO: 23 - DNA, 20 bp, Artificial Sequence
  TLR4 antisense primer
gaaggcgatacaattccacc

SEQ ID NO: 24 - DNA, 25 bp, Artificial Sequence
  CCL3 sense primer
cggaagattccacgccaattcatcg

SEQ ID NO: 25 - DNA, 27 bp, Artificial Sequence
  CCL3 antisense primer
cagatctgccggtttctcttagtcagg

SEQ ID NO: 26 - DNA, 22 bp, Artificial Sequence
  CCL4/MIP-1 sense primer
cagctctgtgcaaacctaaccc

SEQ ID NO: 27 - DNA, 20 bp, Artificial Sequence
  CCL4/MIP-1 antisense primer
aaccctggagcacagaaggc

SEQ ID NO: 28 - DNA, 22 bp, Artificial Sequence
  CCL5/ RANTES sense primer
tgtttgtcactcgaaggaaccg

SEQ ID NO: 29 - DNA, 23 bp, Artificial Sequence
  CCL5/ RANTES antisense primer
tgggggtcagaatcaagaaaccc

SEQ ID NO: 30 - DNA, 23 bp, Artificial Sequence
  CXCL10 sense primer
atgacgggccagtgagaatgagg

SEQ ID NO: 31 - DNA, 20 bp, Artificial Sequence
  CXCL10 antisense primer
gcactgcacaagaagatgcg